SEQUENCE LISTING (1 sequence)

SEQ ID NO: 1 - protein, 11 aa, synthetic construct
MGCVQCKDKEA

(19) United States
(12) Patent Application Publication
(10) Pub. No.: US 2025/0145676 A1
(43) Pub. Date: May 8, 2025

(54) FUNCTIONAL EXPRESSION OF ECTOPIC TMC1 AND TMC2 IN HETEROLOGOUS CELLS

(71) Applicant: The Hong Kong University of Science and Technology, Hong Kong (CN)

(72) Inventors: Pingbo HUANG, Hong Kong (CN); Yixuan CHEN, Hong Kong (CN)

(21) Appl. No.: 18/937,387

(22) Filed: Nov. 5, 2024

Related U.S. Application Data
(60) Provisional application No. 63/597,313, filed on Nov. 8, 2023.

(51) Int. Cl. C07K 14/47; G01N 33/50

(52) U.S. Cl. CPC C07K 14/4702; G01N 33/5044; C07K 2319/033; C07K 2319/40

(57) ABSTRACT

The subject invention pertains methods for the ectopic expression of a transmembrane protein lacking plasma membrane expression in a heterologous cell line. In embodiments, transmembrane proteins Fyn-TMC1 and/or Fyn-TMC2 are fused to a membrane-anchoring motif, a Fyn lipidation tag, which provides a signal for myristoylation and palmitoylation, allowing further research on these proteins, including but not limited to their function, structure, and drug screening.

### CROSS-REFERENCE TO RELATED APPLICATION

The subject application claims the benefit of U.S. Provisional Application Ser. No. 63/597,313, filed Nov. 8, 2023, which is incorporated herein by reference in its entirety.

### SEQUENCE LISTING

The Sequence Listing for this application is labeled “HKUS-198X-SeqList.xml” which was created on Oct. 30, 2024 and is 1,855 bytes. The entire contents of the sequence listing is incorporated herein by reference in its entirety.

### BACKGROUND OF THE INVENTION

### 1.1 General Introduction

The mechanontransduction (MT) channel in cochlear and vestibular hair cells plays a pivotal role in converting the mechanical stimulation of sound and head movements into electrochemical signals that can be processed by the central nervous system. Unraveling the molecular identity of this MT channel in humans and other vertebrates has long been regarded as the “Holy Grail” in hearing research. TMC1 and TMC2 (TMC1/2) have been identified as the pore-forming subunit of the MT channel (Kim & Fettiplace 2013; Pan et al. 2013; Pan et al. 2018; Clark et al. 2023; Jeong et al. 2022). However, the functional expression of TMC1/2 in heterologous cells, critical for unequivocal identification of them as bona fide pore-forming subunit of the MT channel, has proved challenging because ectopic TMC1/2 are known to get trapped in the endoplasmic reticulum (ER) (Yu et al. 2020; Labay et al. 2010; Zhao et al. 2014).

In a study aimed at investigating the function of ectopic TMC1/2, truncated TMC1 from green sea turtle (CmTMC1) and TMC2 from budgerigar (MuTMC2) expressed in insect Sf9 cells were reconstituted in artificial liposomes, where the truncated CmTMC1 or MuTMC2 proteins reportedly formed an MS channel (Jia et al., 2020). However, that study, featured several limitations: (1) the mechanical responses of TMC1/2 single channels in membrane patches showed no clear open-close transitions and the responses cannot be readily distinguished from the noise resulting from suction-induced membrane perturbation, a view shared by several experts in the MS channel field; (2) the TMC1/2 channels used in the study are not full-length proteins. (3) the technique that was utilized, i.e., reconstitution in liposomes, cannot be scaled up and used for drug screening. Therefore, there is a need for a scalable and reliable method to express proteins lacking plasma membrane expression in a heterologous cell line.

### BRIEF SUMMARY OF THE INVENTION

The subject invention pertains to a novel method for expressing proteins lacking plasma membrane expression in a heterologous cell line.

In one aspect, the subject invention discloses a novel method for the ectopic expression of a transmembrane protein in a heterologous cell line, comprising adding a Fyn, or a similar lipidation tag, to the N-terminus thereby promoting the protein expression on the cell surface.

In certain embodiments, the transmembrane protein may include, but is not limited to, Fyn-TMC1 and/or Fyn-TMC2 proteins, fused to an 11-residue membrane-anchoring motif (MGCVQCKDKEA) (SEQ ID NO:1) derived from the Src kinase Fyn protein, named Fyn tag or Fyn lipidation tag, at the N-terminus of mTMC1/2 or hTMC1/2. In certain embodiments, the membrane-anchoring motif provides a signal for myristoylation and palmitoylation. In preferred embodiments, Fyn-mTMC1/2-mCherry expressed in PK-HEK293T cells are expressed on the cell surface.

In another aspect, the subject invention discloses a novel functional and/or structural assay utilizing the Fyn-mTMC1/2 channel for functional and structural studies of the MT channel complex. In certain embodiments, the Fyn-mTMC1/2+mTMIE channel can be used for an in vitro functional assay to study the biophysical and pharmacological properties of the MT channel.

In certain embodiments, the Fyn-TMC1/2 and TMIE channel can be utilized as a medium or high throughput assay for screening specific high-affinity drugs targeting TMC1/2 and other MT-complex components for basic research and clinical application.

### BRIEF DESCRIPTION OF THE DRAWINGS

FIGS. 1A-1B illustrate Fyn tag driving ectopic mouse TMC1/2 to the cell-surface of PK-HEK293T cells. FIG. 1A shows a schematic of TMC1/2 with and without Fyn tag. Fyn-TMC1/2 localize in the plasma membrane (top) and TMC1/2 in the ER (bottom). The C-terminal mCherry tag facilitates visual identification of TMC1/2 in functional assays. FIG. 1B shows confocal images of PK-HEK293T cells expressing mTMC1/2-mCherry (red) with or without Fyn tag. EGFP-CAAX (green) is used as a cell-surface membrane marker; Hoechst 33258 staining shows the nuclei (blue). The last column shows an enlargement of the area indicated by the white arrowhead in the 3rd column, and the yellow arrowheads indicate overlapping of Fyn-TMC1/2-mCherry with EGFP-CAAX. Scale bars, 10 μm except for 5 μm in the last column.

FIGS. 2A-2B illustrate patch clamp assay and calcium imaging assay. All the experiments were performed in PK-HEK293T cells. FIG. 2A shows representative traces of MS whole-cell currents in cells expressing Fyn-mTMC1/2-mCherry with or without mTMIE-HA in patch clamp assay. mTMIE-HA was constructed in pcDNA-IRES-EGFP vector. Cells were mechanically stimulated through stepwise indentation (1 μm/step, top of current traces) at a 45° angle. mTMC1-mCherry, mTMC2-mCherry, empty vector, or mTMIE-HA alone failed to generate any MS currents. Fyn-TMC1-mCherry and Fyn-TMC2-mCherry generated moderate MS whole-cell currents. Addition of mTMIE robustly increased Fyn-mTMC1/2-mCherry-mediated MS currents. Vm=−80 mV in all panels except for-90 mV in the panel of mTMIE-HA alone. FIG. 2B shows representative calcium response traces of PK HEK293T cells transfected with Fyn-mTMC2-mCherry and mTMC2-mCherry in calcium imaging assay. Fluo-4-AM was used as calcium indicator. Tested cells were visually identified by the expression of mCherry and mechanically stimulated through stepwise indentation (2 μm/step, black trace on the top). Fluorescent intensity is expressed in arbitrary unit.

FIGS. 3A-3C illustrate Fyn-mTMC2-mCherry+mTMIE cation-selective and DHS-sensitive channel. FIG. 3A-3B shows IV relation of whole-cell MS currents mediated by Fyn-mTMC2-mCherry+mTMIE. Upper panel, shows representative traces of whole-cell MS currents in a PK-HEK293 cell co-expressing Fyn-mTMC2-mCherry and mTMIE. Lower panel shows IV relation of 4 experiments similar to the upper panel. The ion concentrations in the bath and pipette solutions are symmetrical in Panel A and asymmetrical in Panel B. The reversal potentials in all experiments in Panel B are positive, indicating a cation-selective channel.

FIG. 3C shows DHS-sensitivity of the Fyn-mTMC2-mCherry+mTMIE channel. Left panel shows a representative trace of whole-cell MS currents before and after 2 mM DHS in the bath (blue). Right panel shows summary data of 4 experiments similar to the left panel. **, p=0.0044. Orange line in all panels, indentation. Paired t-test was used for statistical analysis.

### BRIEF DESCRIPTION OF THE SEQUENCES

SEQ ID NO: 1: MGCVQCKDKEA (Fyn lipidation tag)

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X+10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

The term “high affinity” means that the agent strongly binds to one or more epitopes of a selected target protein molecule or cellular target in an in vivo or ex vivo setting.

The term “transmembrane protein” is a protein that extends into or through the cell membrane's lipid bilayer; it can span the membrane once, or more than once. Examples of transmembrane proteins include the insulin receptor, adenylate cyclase, and intestinal-brush border esterase.

The term “Fyn-mTMC1/2” comprises the use of Fyn-mTMC1 and/or Fyn-mTMC2 proteins, alone or in combination.

Other features and advantages of the invention will be apparent from the following description of the preferred embodiments thereof, and from the claims.

All references cited herein are hereby incorporated by reference in their entirety.

The subject invention relates to a novel method for the ectopic expression of a transmembrane protein trapped in the intracellular compartments, comprising adding a Fyn or a similar lipidation tag to the N-terminus, thereby promoting the protein expression on the cell surface and facilitating the study of its function, structure, and/or to use it to screen compounds for new drug discovery.

In one aspect of the subject invention, a Fyn lipidation tag is added to the N-termini of mouse TMC1/2 (mTMC1/2) and/or human TMC1 (hTMC1), driving the proteins to the cell surface of cell lines, including, but not limited to, PIEZO1-knockout HEK293T (PK-HEK293T) cells, HEK293 cells, 3T3 cells, and COS7 cells.

In certain embodiments, Fyn-mTMC1/2 alone, ectopically expressed, for example, in PK-HEK293T cells, form mechanosensitive (MS) channels without mTMIE, a crucial component of MT channel. In certain embodiments, Fyn-mTMC1/2 alone, ectopically expressed, for example, in PK-HEK293T cells, form mechanosensitive (MS) channels with the concurrent expression of mTMIE, a crucial component of MT channel. In preferred embodiments, the channel activities are highly stimulated by the co-expression of mTMIE. In embodiments, the Fyn-mTMC1/2+mTMIE channel of the subject invention recapitulates the biophysical and pharmacological properties of the MT channel.

In another aspect of the subject invention, disclosed hereby is a method for functional and structural studies of the MT channel complex utilizing an in vitro assay comprising Fyn-TMC1/2 and TMIE. In some embodiments, the in vitro assay can be used as a medium for screening specific high-affinity drugs targeting TMC1/2 and other MT-complex components, which are in dire scarcity and vital for basic research and clinical application. In certain embodiments, the in vitro assay can be used as a medium or high throughput assay. In preferred embodiments, adding Fyn, or a similar lipidation tag to the N-termini of transmembrane proteins or other ectopically expressed proteins that are trapped in the intracellular compartments, promotes their expression on the cell surface, thereby facilitating the study of their function, structure, and drug screening.

In certain embodiments, the method comprises an assay including, but not limited to, a transmembrane protein functional assay or transmembrane protein structural assays, or a medium throughput assay for screening specific high-affinity drugs interacting with the transmembrane protein, a high throughput assay for screening specific high-affinity drugs interacting with the transmembrane protein, a basal intracellular calcium signal assay, a calcium imaging assay for determining changes in basal intracellular calcium signal after addition of a pharmaceutical compound, a drug binding affinity to the transmembrane protein, or measuring activity of the transmembrane protein after exposure of novel aminoglycoside antibiotics utilizing patch-clamp; or a drug screening assay for high-affinity drugs.

In certain embodiments, Fyn-TMC1 and/or Fyn-TMC2 are fused to a Fyn tag, i.e., an 11-residue membrane-anchoring motif (MGCVQCKDKEA) (SEQ ID NO:1) derived from the Src kinase at the N-terminus of mTMC1/2. The Fyn lipidation tag is used as a signal for myristoylation and palmitoylation. In certain embodiments, cells expressing Fyn-mTMC1/2-mCherry include, but are not limited to, PIEZO1-knockout HEK293T (PK-HEK293T) cells, HEK293 cells, 3T3 cells, and COS7 cells. In preferred embodiments, Fyn-mTMC1/2-mCherry proteins are expressed on the cell surface.

In certain embodiments, the subject invention provides effective functional assays to characterize the function of Fyn-mTMC1/2 channel in cells, including, but not limited to, PK-HEK293T. Other cells include, but are not limited to, PIEZO1-knockout HEK293T (PK-HEK293T) cells, HEK293 cells, 3T3 cells, and COS7 cells. In certain embodiments, functional assays comprise poking the cell membrane, including, but not limited to, patch clamp and calcium imaging assays. In certain embodiments, mTMIE is added to ectopic Fyn-mTMC1/2 forming an MS channel to increase their activity. The Fyn-mTMC1/2+mTMIE channel can be used as a model having the biophysical and pharmacological properties of the MT channel. In preferred embodiments, the in vitro functional assay of the subject invention comprising Fyn-TMC1/2 and TMIE greatly facilitates functional and structural studies of the MT channel complex and can be utilized as a medium or high throughput assay for screening specific high-affinity drugs targeting TMC1/2 and other MT-complex components.

In certain embodiments, the ectopic transmembrane protein includes, but is not limited to, a receptor, an ion channel, a transporter, a transfer protein, a flippase, a floppase, a neurotransmitter, an enzyme, a scramblase, an ATPase, an drug/metabolite transporter, an aquaporin, a chloride channel, or a transmembrane protein with unknown function, or any combinations thereof.

In certain embodiments, the method of the subject invention comprises further assays for analyzing functional and structural properties of transmembrane proteins, including, but not limited to, calcium imaging assay, drug binding affinity assay, patch clamp, electrophoresis, crystallography, spectrofluorimetry, protein solubilization, surface hydrophobicity, thermal properties, molecular interaction techniques, spectroscopy (such as, for example, UV-Vis spectroscopy, Fourier Transform Infrared (FTIR) spectroscopy, Raman spectroscopy, Circular Dichroism (CD) spectroscopy, fluorescence spectroscopy, Nuclear Magnetic Resonance (NMR) spectroscopy, or any other assay known in the art.

### Materials and Methods

### Cell Culture and Transfection

PK-HEK293T (PIEZO1-knockout HEK293T) cell line was generously provided by Dr. Bailong Xiao (Tsinghua University). HEK293T, 3T3, and COS7 cell lines were purchased from American Type Culture Collection (ATCC); the cell lines were presumably authenticated by ATCC and were not further authenticated in this study. All cell lines were cultured in Dulbecco's Modified Eagle Medium (DMEM) supplemented with 10% fetal bovine serum and 100 mg/L ampicillin in an atmosphere of 95% air/5% CO2 at 37° C. Cells were transfected using polyethylenimine (23966-2; Polysciences), as previously described (Yu et al. 2020).

### Fluorescence Imaging

Cells were digested with trypsin and re-plated on specialized confocal dishes. For live imaging, cells were examined under a confocal microscope (SP8 LIGHTNING, Leica Microsystems) at room temperature after 24 h of transfection with various plasmids and subsequent change of the culture medium to Opti-MEM (31985088, Thermo Fisher Scientific). Cell nuclei were stained by incubating cells with Hoechst 33258 dye for 15 min and then washing twice with Opti-MEM before imaging.

### Whole-Cell Poking and Patch-Clamp Assays

Briefly, PK-HEK293T cells were incubated for 24 h after transfection before performing the whole-cell patch-clamp assay at room temperature. For co-transfection of two plasmids, the w/w ratio of TMC1/2 plasmids and TMIE plasmid was 3:1. The bath solution contained (in mM) 137 NaCl, 5.8 KCl, 0.7 NaH2PO4, 10 HEPES, 1.3 CaCl2, 0.9 MgCl2, 5.6 glucose (pH 7.3 with NaOH); the pipette solution contained (in mM) 137 CsCl, 0.1 EGTA, 10 HEPES, 3.5 MgCl2 (pH 7.3 with CsOH). The recording glass pipettes (1B150-4 Borosilicate Glass Capillaries, World Precision Instruments) were pulled and polished using a pipette puller (DMZ, Zeitz Instruments) and featured a resistance of 3-5 MQ. The mechanical stimulus was applied by poking cells with a fire-polished glass probe with a tip diameter of 2-3 μm. The displacement of the glass probe was controlled by a piezoelectric system (P-601 PiezoMove Linear Actuator/E-625 Piezo Servo Controller, Physik Instrumente) that was under the command of a digitizer (Axon™ Digidata® 1550B, Axon Instruments). The glass probe was set against the cell membrane at a 45° angle and each step of the increment was 1 μm. For testing the cation selectivity of the MS current, the pipette solution was changed to (in mM) 27 CsCl, 0.1 EGTA, 10 HEPES, 230 sucrose, 3.5 MgCl2 (pH 7.3 with CsOH). For testing sensitivity to DHS, the MT-channel blocker was added at a final concentration of 2 mM into the bath. Data were acquired using an Axopatch 200B amplifier and Axon™ Digidata® 1550B with Axon Clampex 10.7 software. The data were sampled at 10 kHz and filtered at 2 kHz. The reversal potentials were calculated using Vpipette at zero current with correction for the junction potential of the pipette and bath solutions (+1.2 mV for whole cell recording). Data were analyzed with Clampfit 10.7 (Molecular Devices). For the measurement of the inactivation time constant, all current responses, except for traces with a noticeable shift in baseline, were individually calculated and averaged.

### Calcium Imaging Assays

Fluo-4-AM (F14201, Thermo Fisher Scientific) was used as the calcium indicator (excitation: 494 nm; emission: 506 nm). After transfection, PK-HEK293T cells were incubated for 24 h, washed once with PBS, and then incubated with 5 μM Fluo-4-AM in Opti-MEM at 37° C. in the dark for 30 min. After washing once with PBS and incubating for another 30 min in Opti-MEM, cells were imaged in the bath solution used for whole-cell patch-clamp assays. Indentation was used to mechanically activate Fyn-mTMC2 channels in PK-HEK293T cells and induce a rise in intracellular calcium. Calcium signals were captured with an EMCCD camera (Andor, Oxford Instruments) and analyzed using NIS-Elements AR software (Nikon) and ImageJ.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

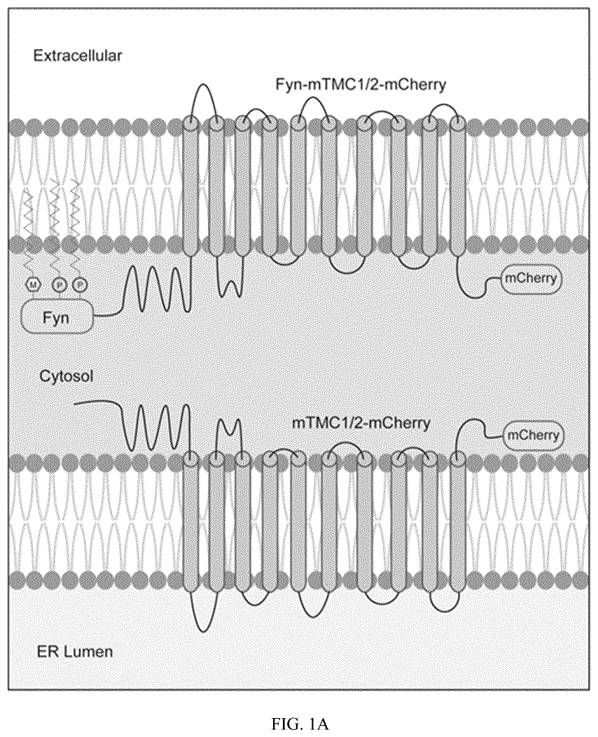

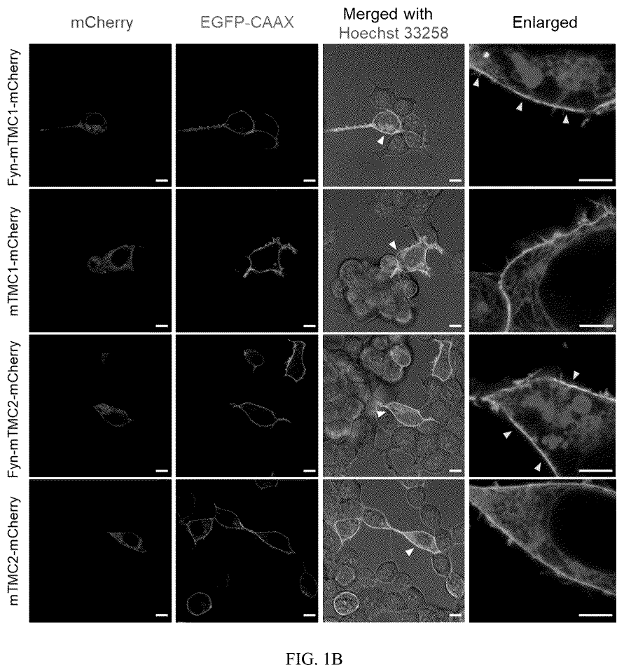

EXAMPLE 1—We engineered Fyn-TMC1 and Fyn-TMC2 by fusing an 11-residue membrane-anchoring motif (MGCVQCKDKEA) (SEQ ID NO:1) derived from the Src kinase Fyn protein, which we named Fyn tag, at the N-terminus of mTMC1/2 (FIG. 1A). This motif serves as a signal for myristoylation and palmitoylation, which aid stable anchoring of proteins to the plasma membrane and thereby promotes their cell surface expression (McCabe & Berthiaume 1999). Remarkably, Fyn-mTMC1/2-mCherry expressed in PK-HEK293T cells reached the cell surface, as evidenced by their co-localization with EGFP-CAAX, a widely used plasmalemmal marker, in confocal microscopy analysis (FIG. 1B).

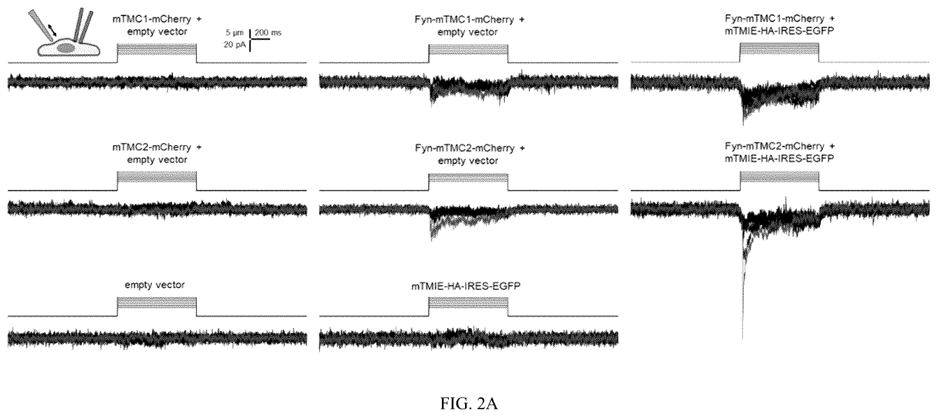

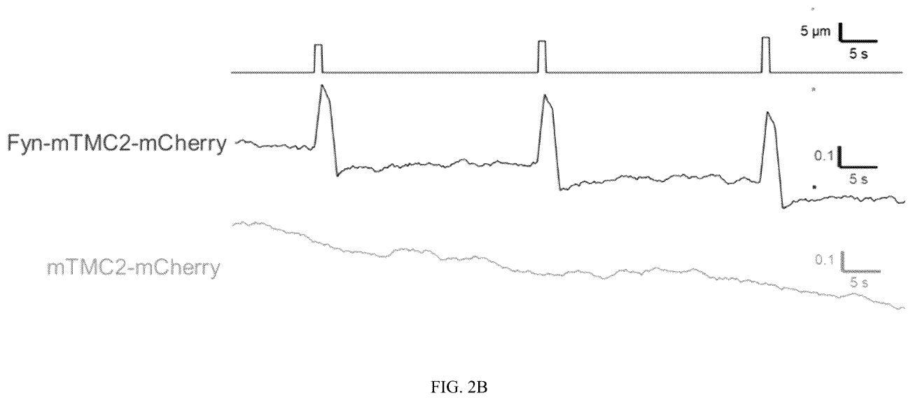

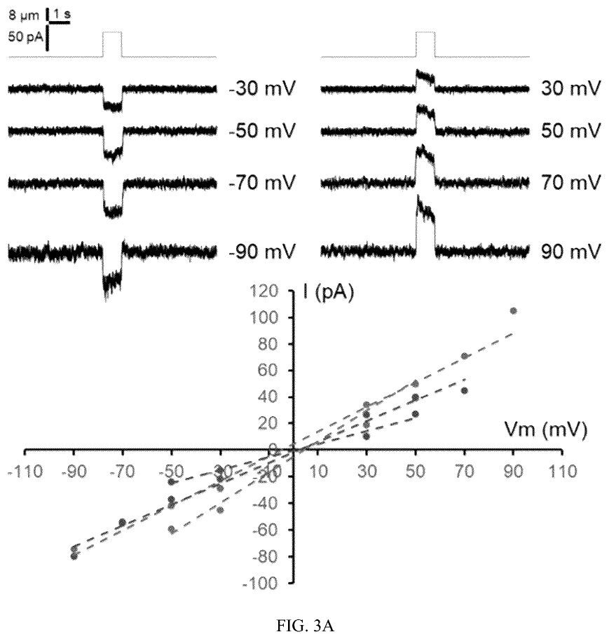

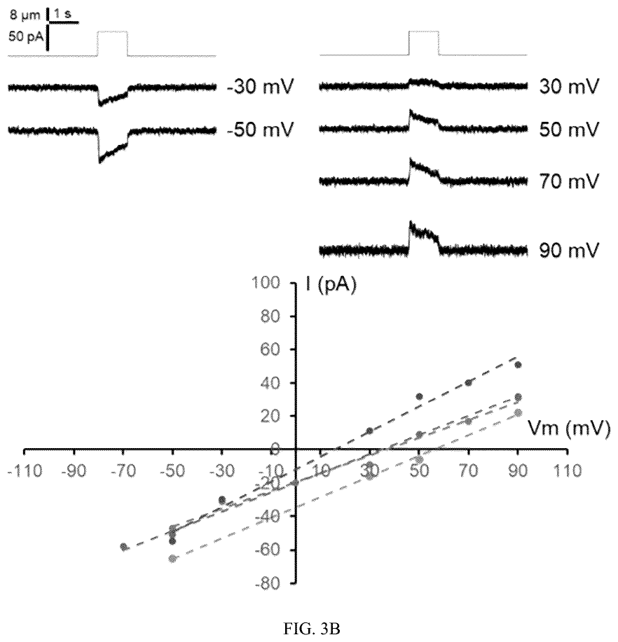

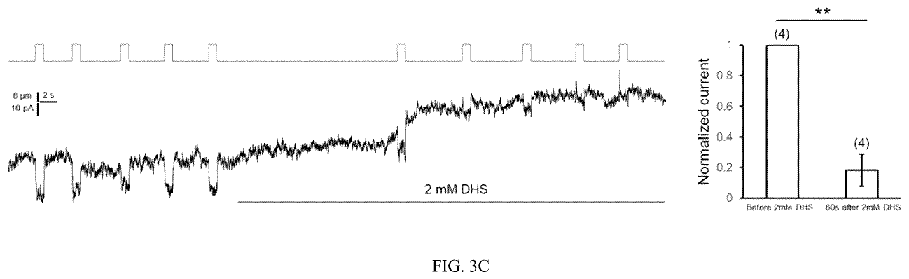

We established effective functional assays in PK-HEK293T cells, to characterize the function of Fyn-mTMC1/2 channel by poking the cell membrane, including using patch clamp and calcium imaging assays. We confirm that ectopic Fyn-mTMC1/2 alone form MS channels whose activities are highly stimulated by mTMIE (FIGS. 2A-2B). Besides, the Fyn-mTMC1/2+mTMIE channel recapitulated biophysical and pharmacological properties of the MT channel (FIGS. 3A-3C). Our in vitro functional assay of Fyn-TMC1/2 and TMIE greatly facilitate subsequent functional and structural studies of the MT channel complex and can be scaled up to a medium or high throughput assay for screening specific high-affinity drugs targeting TMC1/2 and other MT-complex components.

EXAMPLE 2—PK-HEK293T cells are transiently transfected or prepared as stable cell lines to express the Fyn-TMC1/2 channel with or without the co-expression of TMIE to potentiate their channel activity. These PK-HEK293T cells are loaded with a calcium indicator such as Fluo-4-AM. In one scenario, the Fyn-TMC1/2 channel has high basal activity, leading to the mediation of extracellular calcium influx. This allows for differentiation between the basal intracellular calcium signal of cells expressing the Fyn-TMC1/2 channel and those that do not express them. Alternatively, when the Fyn-TMC1/2 channel does not have high basal activity, mechanical stimulation, such as poking the cell membrane, applying ultrasonication, or using magnetic tweezers, is employed to activate the Fyn-TMC1/2 channel and induce intracellular calcium response. Subsequently, compounds extracted from traditional Chinese medicine are added, and changes in the basal intracellular calcium signal or the cells' MS calcium response are evaluated to screen for agonists or antagonists targeting the TMC1/2 channel. This provides potential drug candidates for the treatment of diseases such as deafness or dizziness caused by vestibular dysfunction.

EXAMPLE 3—HEK293S cells are transiently transfected with an AAV viral vector and cultured in suspension to achieve high expression of Fyn-TMC1/2 channel proteins. The plasma membrane proteins are isolated by using either high-speed density gradient centrifugation or a commercial plasma membrane fraction kit. The Fyn-TMC1/2 channel protein is then preliminarily purified from the plasma membrane protein using an appropriate affinity tag and further purified by size exclusion chromatography. The purified Fyn-TMC1/2 channel protein is mixed with validated drugs to form a complex for the sample preparation of vitrification. Subsequently, cryo-electron microscopy is used to collect single-particle structural data to reconstruct the three-dimensional structure of the Fyn-TMC1/2 channel protein in complex with the drug. This enables the identification of the drug's binding pocket and provide a structural basis for the optimization of drug binding affinity, specificity to the Fyn-TMC1/2 channel, and the design of novel drugs for clinical application.

EXAMPLE 4—It has been reported that aminoglycoside antibiotics such as gentamicin and dihydrostreptomycin (DHS) can enter inner ear hair cells through the MT channel and cause permanent hearing loss, known as ototoxicity, which limits their clinical usage. Considering the growing resistance of bacteria to antibiotics, it is possible to design novel aminoglycoside antibiotics that eliminate ototoxicity by modifying the existing drug molecules based on the way they bind to TMC1/2 and using molecular dynamics method for simulations. These novel antibiotics can be validated by conducting patch clamp assays on the Fyn-TMC1/2 channel expressed in PK-HEK293T cells. With the co-expression of TMIE to enhance the channel activity, the Fyn-TMC1/2 channel generates the whole-cell MS current in response to continuous indentation mechanical stimulation. The newly designed antibiotics with antimicrobial activity can be added to the bath solution to verify if they lose the blocking effect on the Fyn-TMC1/2 channel, compared to the original drugs. This lays the foundation for subsequent animal experiments and the development of novel aminoglycoside antibiotics that eliminate ototoxicity for clinical application.

EXAMPLE 5—Any transmembrane proteins, such as receptors, ion channels, transporters, or scramblases, that lack plasmalemmal expression in heterologous cell lines are manipulated to be ectopically expressed on the cell surface by fusing the Fyn tag or other similar tags that possess myristoylation and palmitoylation modifications at their N-terminus. This allows for further research of these transmembrane proteins on the functional, structural, and drug screening aspects like those described in Examples 1-3, which swill potentially generate economic benefits.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

Embodiment 1. A method for the ectopic expression of a transmembrane protein, the method comprising:
(a) obtaining an ectopic transmembrane protein lacking plasma membrane expression in a heterologous cell line;
(b) expressing the ectopic transmembrane protein on the plasma membrane of a heterologous cell by fusing a tag at the N-terminus, wherein the tag comprises a Fyn lipidation tag or other motif that serves as a signal for myristoylation and palmitoylation modifications;
(c) performing an assay,
wherein the assay comprises:
(i) transmembrane protein functional assay, transmembrane protein structural assays;
(ii) medium throughput assay for screening specific high-affinity drugs interacting with the transmembrane protein, high throughput assay for screening specific high-affinity drugs interacting with the transmembrane protein, basal intracellular calcium signal;
(iii) calcium imaging assay for determining changes in basal intracellular calcium signal after addition of a pharmaceutical compound, drug binding affinity to the transmembrane protein, measuring activity of the transmembrane protein after exposure of novel aminoglycoside antibiotics utilizing patch-clamp; and
(iv) medium or high-throughput drug screening assay for high-affinity drugs, or combinations thereof.

Embodiment 2. The method of embodiment 1, wherein the ectopic transmembrane protein comprises mouse TMC1/2 (mTMC1/2) or human TMC1 (hTMC1).

Embodiment 3. The method of any preceding embodiment, wherein the ectopic transmembrane protein comprises a receptor, an ion channel, a transporter, a transfer protein, a flippase, a floppase, a neurotransmitter, an enzyme, a scramblase, or combinations thereof.

Embodiment 4. The method of any preceding embodiment, wherein the ectopic transmembrane protein is fused with mCherry at the C-terminus to form Fyn-mTMC2-mCherry.

Embodiment 5. The method of any preceding embodiment, wherein Fyn-mTMC2-mCherry is co-expressed with TMIE.

Embodiment 6. The method of any preceding embodiment, wherein the ectopic transmembrane protein comprises Fyn-TMC1/2 channel co-expressed with TMIE.

Embodiment 7. The method of any preceding embodiment, wherein the Fyn lipidation tag comprises a membrane-anchoring motif of SEQ ID NO:1.

Embodiment 8. The method of any preceding embodiment, wherein the ectopic transmembrane protein is transiently expressed.

Embodiment 9. The method of any preceding embodiment, wherein the ectopic transmembrane protein is stably expressed.

Embodiment 10. The method of any preceding embodiment, wherein the heterologous cell line comprises PIEZO1-knockout HEK293T (PK-HEK293T), HEK293, 3T3, or COS7.

Embodiment 11. The method of any preceding embodiment, wherein the transmembrane protein comprises Fyn-mTMC1/2 or Fyn-hTMC1, and is optionally co-expressed with TMIE.

Embodiment 12. The method of any preceding embodiment, wherein the assay is an in vitro assay.

### REFERENCES

Kim KX & Fettiplace R (2013) Developmental changes in the cochlear hair cell mechanotransducer channel and their regulation by transmembrane channel-like proteins. J. Gen. Physiol. 141, 141-8. Available at: http://www.ncbi.nlm.nih.gov/pubmed/23277480 [Accessed Jul. 6, 2017].
Pan B, Geleoc GS, Asai Y, Horwitz GC, Kurima K, Ishikawa K, Kawashima Y, Griffith AJ & Holt JR (2013) TMC1 and TMC2 are components of the mechanotransduction channel in hair cells of the mammalian inner ear. Neuron 79, 504-515. Available at: http://www.ncbi.nlm.nih.gov/entrez/query.fcgi?cmd=Retrieve&db=PubMed&dopt=Citati on&list_uids=23871232.
Pan B, Akyuz N, Liu XP, Asai Y, Nist-Lund C, Kurima K, Derfler BH, György B, Limapichat W, Walujkar S, Wimalasena LN, Sotomayor M, Corey DP & Holt JR (2018) TMC1 Forms the Pore of Mechanosensory Transduction Channels in Vertebrate Inner Ear Hair Cells. Neuron 99, 736-753.e6. Available at: https://doi.org/10.1016/j.neuron.2018.07.033 [Accessed May 28, 2019].
Clark S, Jeong H, Posert R, Goehring A & Gouaux E (2023) Structure of C. elegans TMC-2 complex suggests roles of lipid-mediated subunit contacts in mechanosensory transduction., 1-19.
Jeong H, Clark S, Goehring A, Dehghani-ghahnaviyeh S, Rasouli A, Tajkhorshid E & Gouaux E (2022) Structures of the TMC-1 complex illuminate mechanosensory transduction. Nature, https://doi.org/10.1038/s41586-022-05314-8.
Yu X, Zhao Q, Li X, Chen Y, Tian Y, Liu S, Xiong W & Huang P (2020) Deafness mutation D572N of TMC1 destabilizes TMC1 expression by disrupting LHFPL5 binding. Proc. Natl. Acad. Sci. 117, 29894-29903. Available at: https://www.pnas.org/content/117/47/29894 [Accessed Nov. 25, 2020].
Labay V, Weichert RM, Makishima T & Griffith AJ (2010) Topology of transmembrane channel-like gene 1 protein. Biochemistry 49, 8592-8. Available at: http://www.ncbi.nlm.nih.gov/pubmed/20672865 [Accessed Jul. 6, 2017].
Zhao B, Wu Z, Grillet N, Yan L, Xiong W, Harkins-Perry S & Mü Ller U (2014) TMIE Is an Essential Component of the Mechanotransduction Machinery of Cochlear Hair Cells. Neuron 84, 954-967. Available at: http://dx.doi.org/10.1016/j.neuron.2014.10.041
Jia Y, Zhao Y, Kusakizako T, Wang Y, Pan C, Zhang Y, Nureki O, Hattori M & Yan Z (2020) TMC1 and TMC2 Proteins Are Pore-Forming Subunits of Mechanosensitive Ion Channels. Neuron 105, 310-321.e3. Available at: https://doi.org/10.1016/j.neuron.2019.10.017.
McCabe J B & Berthiaume LG (1999) Functional roles for fatty acylated amino-terminal domains in subcellular localization. Mol. Biol. Cell 10, 3771-3786.

We claim:

1. A method for the ectopic expression of a transmembrane protein, the method comprising:
(a) obtaining an ectopic transmembrane protein lacking plasma membrane expression in a heterologous cell line;
(b) expressing the ectopic transmembrane protein on the plasma membrane of a heterologous cell by fusing a tag at the N-terminus, wherein the tag comprises a Fyn lipidation tag or other motif that serves as a signal for myristoylation and palmitoylation modifications; and
(c) performing an assay,
wherein the assay comprises:
(i) a transmembrane protein functional assay or transmembrane protein structural assay; or
(ii) a medium throughput assay for screening specific high-affinity drugs interacting with the transmembrane protein, a high throughput assay for screening specific high-affinity drugs interacting with the transmembrane protein, or a basal intracellular calcium signal assay;
(iii) a calcium imaging assay for determining changes in basal intracellular calcium signal after addition of a pharmaceutical compound, a drug binding affinity to the transmembrane protein, or measuring activity of the transmembrane protein after exposure of novel aminoglycoside antibiotics utilizing patch-clamp; or
(iv) a drug screening assay for high-affinity drugs.

2. The method of claim 1, wherein the wherein the ectopic transmembrane protein comprises mouse TMC1/2 (mTMC1/2) or human TMC1 (hTMC1).

3. The method of claim 1, wherein the ectopic transmembrane protein comprises a receptor, an ion channel, a transporter, a transfer protein, a flippase, a floppase, a neurotransmitter, an enzyme, or a scramblase, or any combination thereof.

4. The method of claim 2, wherein the ectopic transmembrane protein is fused with mCherry at the C-terminus to form Fyn-mTMC2-mCherry.

5. The method of claim 4, wherein Fyn-mTMC2-mCherry is co-expressed with TMIE.

6. The method of claim 2, wherein the ectopic transmembrane protein comprises Fyn-TMC1/2 channel co-expressed with TMIE.

7. The method of claim 1, wherein the Fyn lipidation tag comprises a membrane-anchoring motif of SEQ ID NO:1 (MGCVQCKDKEA).

8. The method of claim 1, wherein the ectopic transmembrane protein is transiently expressed.

9. The method of claim 1, wherein the ectopic transmembrane protein is stably expressed.

10. The method of claim 1, wherein the heterologous cell line comprises PIEZO1-knockout HEK293T (PK-HEK293T), HEK293, 3T3, or COS7.

11. The method of claim 1, wherein the transmembrane protein comprises Fyn-mTMC1/2 or Fyn-hTMC1, and is optionally co-expressed with TMIE.

12. The method of claim 1, wherein the assay is an in vitro assay.
